(19) United States
(12) Patent Application Publication
(10) Pub. No.: US 2022/0184100 A1
(43) Pub. Date: Jun. 16, 2022

(54) ENHANCED D VITAMIN NUTRACEUTICAL COMPOSITIONS AND METHODS FOR MAKING AND USED SAME

(71) Applicant: MM Plant Co. LLC, Houston, TX (US)

(72) Inventor: Mark Vorderbruggen, Houston, TX (US)

(73) Assignee: MM Plant Co. LLC, Houston, TX (US)

(21) Appl. No.: 17/546,982

(22) Filed: Dec. 9, 2021

Related U.S. Application Data
(60) Provisional application No. 63/126,195, filed on Dec. 16, 2020.

(51) Int. Cl. A61K 31/592; A61K 36/074; A61K 36/068; A61K 36/07; A61K 36/481; A61K 36/344; A61K 36/484; A61K 36/53; A61K 36/536; A61K 36/185; A61K 36/537; A61K 36/235; A61K 36/8962; A61K 36/9068; A61K 36/28; A61K 36/258; A61K 36/284; A61K 36/232; A61K 36/638; A61K 36/41

(52) U.S. Cl. CPC A61K 31/592; A61K 2236/17; A61K 36/068; A61K 36/07; A61K 36/481; A61K 36/344; A61K 36/484; A61K 36/53; A61K 36/536; A61K 36/185; A61K 36/537; A61K 36/235; A61K 36/8962; A61K 36/9068; A61K 36/28; A61K 36/258; A61K 36/284; A61K 36/232; A61K 36/638; A61K 36/41; A61K 2236/15; A61K 36/074

(57) ABSTRACT

D vitamins enhanced nutraceutical compositions including one or more UV light treated, dried, and comminuted fungus materials and nutraceutical compositions including one or more UV light treated, dried, and comminuted fungus materials and one or more dried and comminuted herb materials and methods for making and using same.

### RELATED APPLICATIONS

This application claims the benefit of and priority to U.S. Provisional Patent Application Ser. No. 63/126,195 filed Dec. 16, 2020 (16 Dec. 2020).

### BACKGROUND OF THE DISCLOSURE

### 1. Field of the Disclosure

Embodiments of the present disclosure relate to increased D vitamins nutraceutical compositions and methods for making and using same.

In particular, embodiments of the present disclosure relate to increased D vitamins nutraceutical compositions and methods for making and using same, wherein the nutraceutical compositions include one or more fungus materials irradiated with UV light, dried, and comminuted to form a powdered increased D vitamins fungus material and the methods include irradiating the one or more fungus materials with UV light to form a UV treated fungus material, drying the UV treated fungus material to form a UV treated and dried fungus material, and comminuting the UV treated, dried fungus material to form a powdered UV treated and dried fungus material. In certain embodiments, the methods also include forming powdered UV treated and dried fungus material into a desired dose format. In other embodiments, the methods also include packaging the dose format into user packages. In other embodiments, the methods also include packaging the user packages into into bulk packages for shipment.

### 2. Description of the Related Art

Sufferers of auto-immune diseases such as Lupus, rheumatoid arthritis, and Hashimoto's Disease are often rendered more susceptible to infections because the common treatment for these issues involves suppressing the immune system. Recipients of organ transplants have similar concerns in regards to their immune system. Many OTC herbal immune system builders include elderberry, echinacea, and other plants known to increase the production of “killer T cells” and other macrophages by the immune system. This renders these OTC products unusable by those with auto-immune disorders as those killer T cells are the ones improperly attacking their own tissues.

It has been shown that there are other ways to help prevent infections or fight infections without activation of killer T cells. These include taking compounds or herbs which block entry of viruses into cells, interfere with the replication of viruses inside cells, and “poisoning” potentially harmful bacteria when viruses enters the body. These protective mechanisms are believed safe for use even with those with overactive immune systems.

Examples of herbal products using this main mechanism of action include plants such as Prunella vulgaris, Artemisia annua, Chinchona, and other similar plants.

Antibacterial and antiviral properties have also been discovered in mushrooms. Shiitake, maitake, and Reishi are just some commonly-used mushrooms proven to have antiviral and antibacterial activity. Their general mechanism of action is to interfere with viral replication inside cells and/or disrupt the viral protein coats, rending them incapable maintain the structural integrity needed to enter and infect cells.

Vitamins C and Ds are known to be important parts of the human immune system. Even though vitamin C is not synthesized or stored by our bodies, most people aren't deficient in it. People with proper amounts of Vitamin Ds in their body have been shown to better resist all sorts of infections without triggering killer T cell activity or other attack immune responses. The most common method of increasing vitamin Ds in the body is exposure to sunlight which results in a natural production of these necessary vitamins. The skin cells of those that suffer from Lupus are more susceptible to UV light damage and so they are advised to stay away from sunlight, leading to a deficiency of vitamin Ds in those who have much to gain from it. Few natural foods have high levels of bioavailable vitamin D, with fatty fish such as mackerel and sardines being some of the best. Milk fortified with vitamin D is another source, but many adults are lactose-intolerant, making fortified milk a poor choice.

There are many immune support dietary supplements on the market which focus on activating those parts of the immune system which are detrimental to those with autoimmune diseases. There are dietary supplements consisting of individual herbs or mushrooms that are offered to fight infections that may or may not be useful for those with immune system issues.

Thus, there is still a need in the art for dietary supplement formulations and methods for making and administering them that are designed specifically for those with autoimmune diseases where the formulation may help prevent bacterial and/or viral infections from occurring or reducing the duration of bacteria/viral infections and/or diseases without triggering the autoimmune disease “self-attacks”.

### SUMMARY OF THE DISCLOSURE

Embodiments of this disclosure provide nutraceutical compositions having antiviral and/or antibacterial activity, wherein the nutraceutical compositions include one or more raw fungus materials treated with UV light having an intensity and a wavelength or a wavelength range for an exposure period, at a temperature, at a pressure sufficient, and a moisture content to increase D vitamins content of the one or more fungus materials. The UV treated fungus material is then dried and comminuted into a powder UV treated and dried fungus material. In certain embodiment, the nutraceutical compositions also include one or more herbs having antiviral and/or antibacterial activity, wherein the herbs are known to disrupt bacterial membranes, coat viral particles, interfere with virus and/or bacterium replication, or otherwise interfere with bacteria and/or virus life cycles without inducing an immune response or inducing a minimal immune response. In other embodiments, the nutraceutical compositions may also include, without limitation, fillers, preservatives, and processing aids needed for the proper manufacturing of the compositions into a dose format or delivery system such as, but not limited to, hard shell capsules, soft gel capsules, tablets, teas, blends, gummies, tinctures, or other common dose formats or delivery systems.

Embodiments of this disclosure provide methods of making the nutraceutical compositions, wherein the methods include supplying one or more raw fungus materials to a UV treating unit, UV treating the one or more raw fungus materials to form a UV treated fungus material. The methods also include drying the UV treated fungus materials to form a UV treated and dried fungus material. The methods also include comminuting the UV treated and dried fungus materials to form a powdered UV treated and dried fungus material. The methods may also include packaging the powdered UV treated and dried fungus material into a dose format. The methods may also include packaging the dose format into a user packages. The methods may also include packaging the user packages into bulk packages for shipping. The methods may also include supplying one or more raw herb materials to a drying unit to form a dried herb material and comminuting the powdered and dried herb material. Optionally, the methods may also include treating the one or more raw herb material to form an optional treated herb material, which is then dried and comminuted to form a treated and dried herb material. In certain embodiments, the methods include mixing the powdered, UV treated, and dried fungus material with the powdered and dried herb material and/or the powdered, treated, and dried herb material before forming the mixture into a dose format or delivery system.

Embodiments of this disclosure provide methods of administering the nutraceutical compositions having antiviral and/or antibacterial activity, wherein the methods include orally administering the powdered, UV treated, and dried fungus material with or without the powdered and dried herb material and/or the powdered, treated, and dried herb material at a dose sufficient to delivery a desired amount D vitamins and/or an amount of therapeutic herbs.

### BRIEF DESCRIPTION OF THE DRAWINGS OF THE DISCLOSURE

The disclosure may be better understood with reference to the following detailed description together with the appended illustrative drawings in which like elements are numbered the same:

FIG. 1A depicts an embodiment of a general apparatus for making the nutraceutical compositions of this disclosure.

FIG. 1B depicts another embodiment of a general apparatus for making the nutraceutical compositions of this disclosure.

FIG. 2 depicts an embodiment of a specific batch apparatus for making the nutraceutical compositions of this disclosure.

FIG. 3 depicts another embodiment of a specific batch apparatus for making the nutraceutical compositions.

FIG. 4A depicts an embodiment of a specific continuous apparatus for making the nutraceutical compositions of this disclosure.

FIG. 4B depicts another embodiment of a specific continuous apparatus for making the nutraceutical compositions of this disclosure.

FIG. 5 depicts an embodiments of a control and power supply system for the apparatus of FIG. 1A.

FIG. 6 depicts an embodiments of a control and power supply system for the apparatus of FIG. 1B.

FIG. 7A depicts a conceptual schematic for making the nutraceutical compositions of this disclosure.

FIG. 7B depicts conceptual schematic for making the nutraceutical compositions of this disclosure.

### DEFINITIONS USED IN THE DISCLOSURE

The term “at least one” means one or more or one or a plurality, additionally, these three terms may be used interchangeably within this application. For example, at least one device means one or more devices or one device and a plurality of devices.

The term “one or a plurality” means one item or a plurality of items.

The term “about” means that a value of a given quantity is within ±20% of the stated value. In other embodiments, the value is within ±15% of the stated value. In other embodiments, the value is within ±10% of the stated value. In other embodiments, the value is within ±5% of the stated value. In other embodiments, the value is within ±2.5% of the stated value. In other embodiments, the value is within ±1% of the stated value.

The term “substantially” means that a value of a given quantity is within±5% of the stated value. In other embodiments, the value is within ±2.5% of the stated value. In other embodiments, the value is within ±2% of the stated value. In other embodiments, the value is within ±1% of the stated value. In other embodiments, the value is within ±0.1% of the stated value.

The term “UV intensity” means the intensity of UV light used to irradiate raw materials herein.

The term “UV frequency” means a specific UV light frequency between 750 THz and 3,000 THz.

The term “UV frequency range” means a specific UV light frequency range, e.g., UV-A light having one or more frequencies between 750 THz and 952 THz, UV-B light having one or more frequencies between 952 THz and 1,071 THz, and/or UV-C having one or more frequencies between 1,071 THz and 3,000 THz.

The term “UV wavelength” means a specific UV light wavelength between 190 nm and 400 nm.

The term “UV wavelength range” means a specific UV light wavelength range, e.g., UV-A light having one or more wavelengths between 315 nm and 400 nm, UV-B light having one or more wavelengths between 290 nm and 315 nm, and/or UV-C having one or more wavelengths between 190 nm and 290 nm.

The term “nm” means nanometer.

The term “THz” means teraHertz.

It should be recognized that all ranges include the end points unless otherwise stated, and all ranges include any and all subranges, regardless of the types of numbers used, e.g., if integers, all integer subranges, if real number, all real number subranges, etc.

### DETAILED DESCRIPTION OF THE DISCLOSURE

The inventor has found that nutraceutical compositions maybe formulated from UV treated, dried, and comminuted antiviral and/or antibacterial fungi, wherein the UV treatment increases an amount of D vitamins in the antiviral and/or antibacterial fungi, the drying locks in the increased D vitamins content of the UV treated antiviral and/or antibacterial fungi, and the comminuting reduces a particle size of the UV treated, dried, and comminuted antiviral and/or antibacterial fungi for forming dose format packages containing the UV treated, dried, and comminuted antiviral and/or antibacterial fungi. The dose size is designed to deliver a sufficient amount of the D vitamins to enhance antiviral and/or antibacterial fungi to reduce effects of virus and/or bacterial infections.

Many culinary fungi produce D vitamins when exposed to sunlight or UV light. The amount of D vitamins produced during sunlight or UV light exposure depends on the surface area available for exposure to the UV light. It has been shown that chopping up the fungi before exposure greatly increases the final D vitamins concentration. Left alone, the D vitamins content of the mushrooms will degrade back to pre-exposure levels within 48 hours. However, if the fungi are quickly or immediately dried and comminuted, they will retain all the induced D vitamins.

Embodiments of this disclosure broadly relate to composition including one or more UV treated, dried, and comminuted (powdered) fungus materials in a dose sufficient to deliver between about 500 international unit (IU) and about 4000 IU of vitamin D2 per day, between about 750 IU and about 3500 IU of vitamin D2 per day, between about 1000 IU and about 3500 IU of vitamin D2 per day, between about 1000 IU and about 3500 IU of vitamin D2 per day, between about 1500 IU and about 3500 IU of vitamin D2 per day, between about 1750 IU and about 3500 IU of vitamin D2 per day, or between about 2000 IU and about 3500 IU of vitamin D2 per day. The comminuted or powdered UV treated and dried fungus materials have particles sizes between about 1 nm and about 1 mm μm, between about 10 nm and about 1 mm, between about 100 nm and about 1 mm, or between about 1 μm and 1 mm. The comminuted and powdered UV treated and dried fungus materials m

In certain embodiments, the compositions of this disclosure also include (a) one or more fungi including powdered UV treated and dried reishi (ganoderma spp.) at a dose of 1 g per day, cordyceps (Cordyceps militaris, Ophiocordyceps sinensis) at a dose of about 1.5 g per day, turkey tail (Gametes versicolor) at a dose about 1 g per day, shiitake (Lentinula edodes) at a dose about 1 g per day and (b) one or more herbs, wherein the one or more herbs comprise astragalus (Astragalus membranaceus) at a dose 3 g per day, Codonopsis (Codonopsis pilosula) at a dose about 1 g per day, licorice (Glycyrrhiza glabra) at a dose about 1 g per day, tulsi (Ocimum tenuiflorum) at a dose about 1 g per day, basil (Ocimum basilicum) at a dose about 1 g per day, self heal (Prunella vulgaris) at a dose about 1 g per day, sweetgum storax (liquidambar spp.) at a dose 1 g per day, oregano at a dose about 1 g per day, sage at a dose about 1 g per day, fennel at a dose about 1 g per day, garlic at a dose between about 1 g and about 5 g per day, lemon balm at a dose about 1.5 g per day, rosemary at a dose between about 0.5 g and about 2 g per day, ginger at a dose between 0.5 g and 2 g per day, Echinacea spp. such as Echinacea angustifolia, Echinacea atrorubens, Echinacea laevigata, Echinacea pallida, Echinacea paradoxa, Echinacea purpurea, Echinacea sanguines, Echinacea serotina, Echinacea simulata, and/or Echinacea tennesseensis at a dose between about 0.4 and about 1 g per day, Elderberry at a dose between about 1 g and about 3 g per day, ginseng at a dose between about 1 g and about 3 g per day, cat's claw (Uncaria tomentosa) at a dose about 1 g per day, spilanthes (Acmella oleracea) at a dose between about 0.5 g and about 2 g per day, atractylodes ovata at a dose between about 1 g and 3 g per day, angelica sinensis at a dose between about 0.5 g and about 2 g per day, Ligustrum lucidum at a dose between about 0.5 g and about 2 g per day, Vitamin C at a dose between about 0.9 g and about 2 g per day, rhodiola at a dose between about 1 g and about 3 g per day, milk thistle or milk thistle extract (silymarin) at a dose between about 0.1 gand about 1 g per day, or mixtures and combinations thereof.

Embodiments of this disclosure broadly relate to apparatuses include a raw fungus material supply unit, a UV treating unit, a drying unit, and a comminuting unit. In certain embodiments, the apparatuses also include a dose formatting unit designed to product dose delivery systems or formats. In other embodiments, the apparatuses also include a user packing unit designed to package the delivery systems or dose forms into user packages including a plurality of specific dose formats or delivery systems. In other embodiments, the apparatuses also include a bulk packaging unit designed to package the user packages into bulk packages including a plurality of user packages.

In certain embodiments, the UV treating unit includes UV irradiating devices or lamps that produce UV-A light, UV-B light, UV-C light or combinations thereof, wherein UV-A light has wavelengths between 315 nm and 400 nm, UV-B light has wavelengths between 290 and 315 nm; and UV-C has wavelengths between 190 nm and 290 nm. In certain embodiments, the treating unit include irradiating devices or lamps that produce UV-B light. The UV treating unit is maintained at a temperature between about 20° C. and 40° C., between about 25 ° C. and 40° C., or between about 30° C. and 40 ° C. and a moisture content between about 50% and about 90%, between about 60% and about 80%, or between about 70% and about 80%. The UV treating time is between 30 minutes and 12 hours, between 45 minutes and 8 hours, between 1 hour and 6 hours, or between about 1 hour and about 4 hours.

In certain embodiments, the drying unit is designed to dry the UV treated fungus material at a temperature, at a pressure, and for a time sufficient to dry the UV treated fungus material, wherein the temperature, pressure and time will depend on the type of drying unit being implemented such information is will known in the art.

Embodiments of this disclosure broadly relate to methods supplying one or more raw fungus materials via a raw fungus material supply unit to a UV treating unit. Treating/irradiating the one or more raw fungi materials with UV light having an intensity and a frequency or a frequency range, at a temperature, and at a moisture content for an irradiating time sufficient to increase and/or maximize vitamin D2 content, a drying unit to dry the UV treated fungus material to form a dried, UV treated fungus material, and a comminuting unit the dried, UV treated fungus material to form a powdered, dried, UV treated fungus material. In certain embodiments, the methods also include forming a dose format or delivery system from the powdered, dried, UV treated fungus material. In other embodiments, the methods also include packing the dose formats or delivery systems into user packages. In other embodiments, the methods also include packing the user packages into bulk packages for shipping. In other embodiments, the methods may also include refrigerating the UV treated mushrooms at between about 2 ° C. to about 5 ° C. for a period of between about 1 to 20 days before drying and comminuting.

### Suitable Components for Use in the Disclosure

### Fungi

Suitable fungi for use herein include, without limitation, Cordyceps sinensis—Cordyceps, fungus growing on caterpillars; Ganoderma lucidum and G. tsugae—reishi, varnished artists conk, fruiting mushroom body; Lentinula edodes—shitake mushroom; or mixtures and combinations thereof. Exemplary examples of such fungi include, without limitation, Lions Mane Mushroom (Hericium erinaceus), Turkey Tail Mushroom (Trametes versicolor), Chaga Mushroom (Inonotus obliquus), Reishi Mushroom (Ganoderma lucidum), Cordyceps Mushroom, Maitake (Grifola frondosa, Hen of the Woods), Shiitake Mushroom (Lentinula edodes), Agaricus blazei, Poria (Wolfiporia extensa, Fú Ling), Tremella fuciformis, Agarikon (Laricifomes officinalis), Phellinus linteus (Sang Hwang Mushroom, Meshima), Boletus frondosus, Cordyceps spp., Fomesfomentarius, Ganoderma lucidum, Grifola frondosa, Hericium erinaceus, Inonotus obliquus, Ophiocordyceps sinensis, Phellinus spp., Polyporus umbellatus, Taiwanofungus camphoratus, Wolfiporia extensa, Aborted Entoloma (Entoloma abortivum), Artists Conk/Artist's Bracket (Ganoderma Applanatum), Beech, Beefsteak mushroom (Fistulina hepatica), Black Trumpet (Craterellus cornucopioides, C. cenerius, C. foetidus), Burnt Matches (Eutypella scoparia), Button mushroom, Cauliflower mushroom, Chanterelle (Cantharellus), Chicken of the Woods (Laetiporus sulphureus), Corn Smut (Ustilago maydis), Cordyceps (Cordyceps militaris), Cremini, Elm Oyster (Hypsizygus ulmarius), Enoki/Enokitake, Fly Amanita, Giraffe Spots (Peniophora albobadia), Gamba mushroom (Thelephora gambajun), Giant Puffball (Calvatia gigantea), Green-spored Lepiota. (Chlorophyllum), Hawk Wings (Sarcodon imbricatus), Hedgehog (Hydnum repandum, Hydnum umbilicatum), Horn of Plenty (Craterellus cornucopiodes), King Oyster, Lactarius or milk mushroom, Lion's Mane Mushrooms (Hericium erinaceus), Lobster Mushrooms (Hypomyces lactifluorum), Maitake (Grifola frondosa or hen of the woods), Matsutake mushroom, Meadow mushroom (Agaricus), Morel (Morchella), Oyster (Pleurotus ostreatus), Pig's Ears (Gomphus clavatus), Porcini/CEP/Penny Bun/Steinpilz/King Bolete (Boletus edulis), Portabello, Reishi (Ganoderma lucidum, Lingzi), Shaggy mane (Coprinus), Shimeji, shitake mushroom (Lentinula edodes), Slippery jack (Suillus, Boletus), Smooth white Lepiota, Snow fungus (Tremellafuciformis), Straw Mushrooms (Volvariella volvacea), Sulfur or Chicken mushroom (Laetiporus), Sweet Coral Clubs (Clavariadelphus cornucopiodes), Termite mushrooms (Termitomyces), Truffles, Turkey Tail, Wine Cap, Wood blewit mushrooms, Wood ear, Agaricus bisporus, Pleurotus species, Lentinula edodes, the Shiitake mushroom, Auricularia auricula-judge, Volvariella volvacea, Flammulina velutipes, Tremella fuciformis, Hypsizygus tessellatus, aka Hypsizygus marmoreus, Bunapi-shimeji, Buna-shimeji, Stropharia rugosoannulata, Cyclocybe aegerita, Hericium erinaceus, Boletus edulis or edible Boletus, Calbovista subsculpta, Calvatia gigantea, Cantharellus cibarius, Craterellus tubaeformis, Clitocybe nuda, Cortinarius caperatus, Craterellus cornucopioides, Grifola frondosa, Gyromitra esculenta, Hericium erinaceus, Hydnum repandum, Lactarius deliciosus, Morchella species such as Morchella conica var deliciosa and Morchella esculenta var. rotunda, Pleurotus species, Tricholoma matsutake, Tuber species, (the truffle) such as Tuber aestivum, Tuber borchii, Tuber brumale, Tuber indicum, Tuber macrosporum, and Tuber mesentericum, Agaricus arvensis (Horse Mushroom), Agaricus silvaticus (Pinewood Mushroom), Amanita caesarea (Caesar's Mushroom), Armillaria mellea (Honey mushroom), Boletus badius (Bay Bolete), Calocybe gambosa (St George's mushroom), Calvatia utriformis (Lycoperdon caelatum), Chroogomphus species (pine-spikes or spike-caps), Clavariaceae species (coral fungus family), Clavulinaceae species (coral fungus family), Coprinus comatus, Corn smut, Cortinarius variicolor, Cyttaria espinosae, Fistulina hepatica (beefsteak polypore or the ox tongue), Flammulina velutipes (Velvet Shank or Winter Fungus), Grifola frondosa (Maitake), Hericium erinaceus (Lion's Mane), Hygrophorus chrysodon, Kalaharituber pfeilii, Auricularia auricula-judae, Lactarius deterrimus (Orange Milkcap), Lactarius salmonicolor, Lactarius salmonicolor, Lactarius subdulcis (mild milkcap), Lactarius volemus (Fishy Milkcap), Laetiporus sulphureus (Sulphur shelf), Leccinum aurantiacum (Red-capped scaber stalk), Leccinum scabrum (Birch bolete), Leccinum versipelle (Orange Birch Bolete/Boletus testaceoscaber), Macrolepiota procera (Parasol Mushroom), Marasmius oreades (Fairy Ring Champignon), Polyporus mylittae, Polyporus squamosus (Dryad's saddle and Pheasant's back mushroom), Ramariaceae species (coral fungus family), Rhizopogon luteolus, Russula species such as R. laeta, Sparassis crispa, Suillus bovinus, Suillus granulatus (Weeping Bolete), Suillus luteus (Slippery Jack), Suillus tomentosus, Tricholoma terreum, Amanita fulva (Tawny Grisette), Amanita rubescens (The Blusher), Coprinopsis atramentaria (Coprinus atramentarius—Common Inkcap), Lactarius spp., Lepista saeva (Field Blewit, Blue Leg, or Tricholoma personatum), Verpa bohemica, or mixtures and combinations thereof.

### Herbs

Suitable herbs for use herein include, without limitation, Prunella vulgaris, artemisi, annua, turmeric, Torreya nucifera, Ocimum basilicum, Canistrocarpus cervicornis, Padina gymnospora, Palisada perforate, Caulerpa racemose, Terminalia chebula, Raoulia australis, Woodforia fruticose, Cordydalis saxicola, Silybum marianum, any other medicinal herb, or mixtures and combinations thereof. Exemplary examples of other medicinal herbs include, without limitation, Agrimony (Agrimonia eupatoria), Alfalfa (Medicago sativa), Aloe (Aloe vera), American Ginseng (Panax quinquefolius), Amla (Phyllanthus emblica), Angelica (Angelica archangelica), Anise (Pimpinella anisum), Arnica (Arnica montana), Ashwagandha (Withania Somnifera), Astragalus (Astragalus membranaceus), Bacopa (Bacopa Monnieri), Bearberry (Arctostaphylos uva-ursi), Bee Balm (Monarda didyma), Bee Pollen (Entomophile pollen), Bilberry (Vaccinium mytillus), Black Cherry (Prunus serotina), Black Cohosh (Cimicifuga racemosa), Boneset (Eupatorium perfoliatum), Borage (Borago officinalis), Boswellia (Boswellia serrata), Buchu (Agathosma betulina), Burdock (Arctium lappa), Butterbur (Petasites hybridus), Calendula (Calendula officinalis), Cascara Sagrada (Frangula purshiana), Catnip (Nepeta cataria), Cat's Claw (Uncaria tomentosa), Cayenne (Capsicum annuum), Chamomile (Matricaria recutita), Chaparral (Larrea tridentata), Chaste Tree (Vitex agnus-castus), Chicory (Cichorium intybus), Cinnamon (Innamonum verum), Clubmoss (Lycopodium clavatum), Comfrey (Symphytum officinale), Cordyceps (Cordyceps sinensis), Dandelion (Taraxacum officinale), Dong Quai (Angelica sinensis), Echinacea spp. (Echinacea angustifolia, Echinacea atrorubens, Echinacea laevigata, Echinacea pallida, Echinacea paradoxa, Echinacea purpurea, Echinacea sanguines, Echinacea serotina, Echinacea simulata, and/or Echinacea tennesseensis), Fo-Ti (Polygonum multiflorum), Ginkgo biloba (Ginkgo biloba), Gotu kola (Centella asictica), Gynostemma (Gynostemma pentaphyllum), Holy Basil (Ocimum sanctum), Kava (Piper Methysticum), Korean Ginseng (Panax ginseng), Lemongrass (Cymbopogon citratus), Licorice Root, Chinese (Glycyrrhiza uralensis), Lycium Fruit (Lycium barbarum), Maca (Lepidiumu meyenii), Milk Thistle (Silybum marianum), Rhodiola (Rhodiola Rosea), Saw Palmetto (Serenoa repens), Schizandra (Schizandra chinensis), Shilajit (Asphaltum), Siberian Ginseng (Eleutherococcus senticosus), Skullcap (Scutellaria lateriflora), St. John's Wort (Hypericum perforatum), Suma (Pfaffia paniculata), Turmeric (Curcuma longa), Valerian Root (Valerian officinalis), Ligustrum lucidum—privet, fruit; ocimum tenuiflorum syn, Ocimum sanctum—holy basil; Panax ginseng—asian ginseng, root; Panax quinquefolius—american ginseng, root; Rhodiola rosea—roseroot, root; Sambucus canadensis and S. nigra—elder, flower and fruit; Schisandra chinensis—wu wei zi, berries; Withania somnifera—ashwagandha, root, or mixtures and combinations thereof.

### Dosing Formats

Suitable modes of administration include oral administration formats including, without limitation, pills, tablets, hard shell capsules, soft gel capsules, drinkables, teas, gummies, other edibles, or any other oral deliverable format.

### Sensors

Suitable sensors for use herein include, without limitation, temperature sensors, chemical sensors such as vitamin D sensors, moisture sensors, or other useful chemical sensors, pressure sensors, exposure sensors, UV light sensors, UV light frequency sensors, UV light intensity sensors, level sensors, feed rate sensors, conveyor belt rate sensors, or any combinations thereof.

### Heaters

Suitable heating units or heater for use herein include, without limitation, resistive heaters, microwave heating units, forced air heating units, conductive heating units, inductive heating units, IR heating units, other radiative heating units, steam heating units, heat transfer units, any other known or yet to be invented heating unit, or any combination thereof.

### Drying Units

Suitable drying units for use herein include, without limitation, forced air drying units, freeze drying units, dry air drying units, IR drying units, microwave drying units, conductive drying units, convention drying units, radiant drying units, dielectric heat drying units, or any combination thereof.

### Comminuting Units

Suitable comminuting units for use herein include, without limitation, grinders, cryogenic-grinders, shredders, crushers, milling units, trituration units, pulverizing units, cutting units, chopping units, vibrating units, sonicating unit, sonic pulverizing units, any other known comminuting units or to be later invented, or any combination thereof.

### Processing Units

Suitable processing units for use in the present disclosure include, without limitation, digital processing units (DPUs), analog processing units (APUs), Field Programmable Gate Arrays (FPGAs), any other technology that may receive motion sensor output and generate command and/or control functions for objects under the control of the processing unit, or any combination thereof

Suitable digital processing units (DPUs) include, without limitation, any digital processing unit capable of accepting input from a plurality of devices and converting at least some of the input into output designed to select and/or control attributes of one or more of the devices. Exemplary examples of such DPUs include, without limitation, microprocessor, microcontrollers, or the like manufactured by Intel, Motorola, Ericsson, HP, Samsung, Hitachi, NRC, Applied Materials, AMD, Cyrix, Sun Microsystem, Philips, National Semiconductor, Qualcomm, or any other manufacture of microprocessors or microcontrollers, or any combination thereof.

Suitable analog processing units (APUs) include, without limitation, any analog processing unit capable of accepting input from a plurality of devices and converting at least some of the input into output designed to control attributes of one or more of the devices or any combination thereof. Such analog devices are available from manufacturers such as Analog Devices Inc.

### Input Devices

Suitable input devices for use herein include, without limitation, motion sensors, keyboards, pointers, microphones, touch screen, touch pads, any other input device, or any combination thereof.

Exemplary examples of motion sensors include, without limitation, optical sensors, acoustic sensors, thermal sensors, optoacoustic sensors, wave form sensors, pixel differentiators, or any other sensor or combination of sensors that are capable of sensing movement or changes in movement, or any combination thereof. Suitable motion sensing apparatus include, without limitation, motion sensors of any form such as digital cameras, optical scanners, optical roller ball devices, touch pads, inductive pads, capacitive pads, holographic devices, laser tracking devices, thermal devices, electromagnetic field (EMF) sensors, wave form sensors, any other device capable of sensing motion, changes in EMF, changes in a wave form, or the like or arrays of such devices or any combination thereof. The sensors may be digital, analog, or any combination of digital and analog units. The motion sensors may be touch pads, touchless pads, touch sensors, touchless sensors, inductive sensors, capacitive sensors, optical sensors, acoustic sensors, thermal sensors, optoacoustic sensors, electromagnetic field (EMF) sensors, strain gauges, accelerometers, pulse or waveform sensor, any other sensor that senses movement or changes in movement, or any combination thereof. The sensors may be digital, analog, or a combination of digital and analog or any other type. For camera systems, the systems may sense motion within a zone, area, or volume in front of the lens or a plurality of lens. Optical sensors include any sensor using electromagnetic waves to detect movement or motion within in active zone. The optical sensors may operate in any region of the electromagnetic spectrum including, without limitation, radio frequency (RF), microwave, near infrared (IR), IR, far IR, visible, ultra violet (UV), or any combination thereof. Exemplary optical sensors include, without limitation, camera systems, the systems may sense motion within a zone, area or volume in front of the lens. Acoustic sensor may operate over the entire sonic range which includes the human audio range, animal audio ranges, other ranges capable of being sensed by devices, or mixtures and combinations thereof EMF sensors may be used and operate in any frequency range of the electromagnetic spectrum or any waveform or field sensing device that are capable of discerning motion with a given electromagnetic field (EMF), any other field, or combination thereof. Moreover, LCD screen(s), other screens and/or displays may be incorporated to identify which devices are chosen or the temperature setting, etc. Moreover, the interface may project a virtual control surface and sense motion within the projected image and invoke actions based on the sensed motion. The motion sensor associated with the interfaces of this disclosure may also be acoustic motion sensor using any acceptable region of the sound spectrum. A volume of a liquid or gas, where a user's body part or object under the control of a user may be immersed, may be used, where sensors associated with the liquid or gas can discern motion. Any sensor being able to discern differences in transverse, longitudinal, pulse, compression or any other waveform may be used to discern motion and any sensor measuring gravitational, magnetic, electro-magnetic, or electrical changes relating to motion or contact while moving (resistive and capacitive screens) could be used. Of course, the interfaces can include mixtures or combinations of any known or yet to be invented motion sensors. The motion sensors may be used in conjunction with displays, keyboards, touch pads, touchless pads, sensors of any type, or other devices associated with a computer, a notebook computer or a drawing tablet or any mobile or stationary device, and/or device, head worn device, or stationary device.

Other exemplary examples of motion sensing apparatus include, without limitation, motion sensors of any form such as digital cameras, optical scanners, optical roller ball devices, touch pads, inductive pads, capacitive pads, holographic devices, laser tracking devices, thermal devices, EMF sensors, wave form sensors, MEMS sensors, any other device capable of sensing motion, changes in EMF, changes in wave form, or the like or arrays of such devices or mixtures or combinations thereof. Other motion sensors that sense changes in pressure, in stress and strain (strain gauges), changes in surface coverage measured by sensors that measure surface area or changes in surface are coverage, change in acceleration measured by accelerometers, or any other sensor that measures changes in force, pressure, velocity, volume, gravity, acceleration, any other force sensor or any combination thereof.

### Output Devices

Suitable output devices for use herein include, without limitation, cathode ray tubes, liquid crystal displays, light emitting diode displays, organic light emitting diode displays, plasma displays, touch screens, touch sensitive input/output devices, audio input/output devices, audio-visual input/output devices, holographic displays and environments, keyboard input devices, mouse input devices, optical input devices, and any other input and/or output device that permits a user to receive user intended inputs and generated output signals, create input signals, or any combination thereof.

### DETAILED DESCRIPTION OF THE DRAWINGS OF THE DISCLOSURE

### General Apparatus Embodiments

### An Embodiment

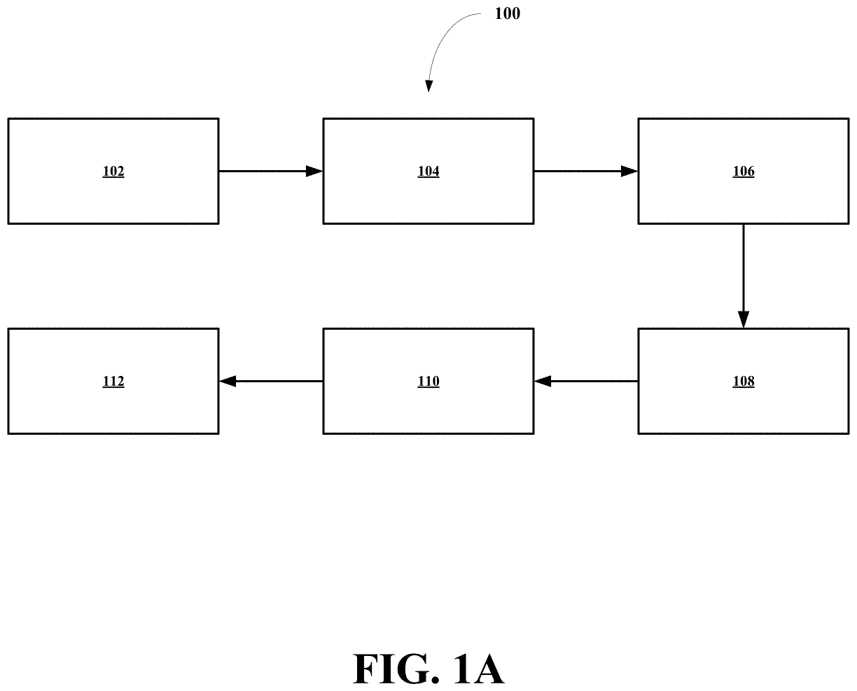

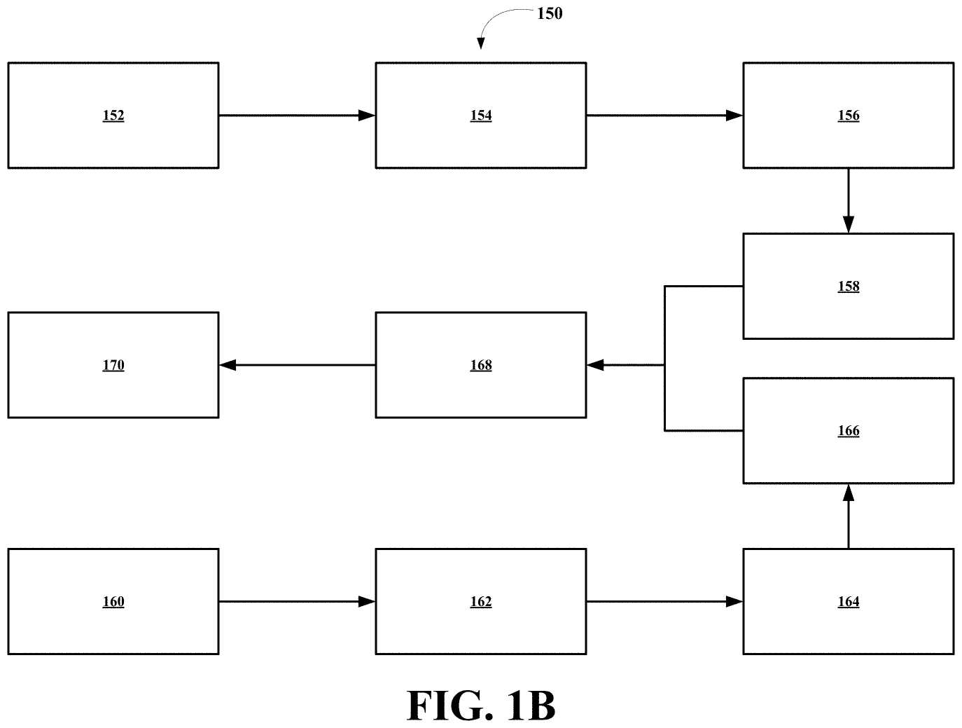

Referring now to FIG. 1A, an embodiment of an apparatus of this disclosure, generally 100, is shown to include a fungi supply unit 102, a fungi UV treating unit 104, a fungi drying unit 106, a fungi comminuting unit 108, a fungi dosing preparation unit 110, and a fungi dose format packaging unit 112.

The fungi UV treating unit 104 is configured to irradiate the fungi with UV light for an adjustable period of time, at an adjustable temperature and at an adjustable pressure. The fungi UV treating unit 104 may also be configured to adjust an intensity of the UV light and a frequency or a frequency range of the UV light. The fungi UV treating unit 104 may also be configured to change the time, temperature, pressure, UV light intensity, and/or frequency or frequency range periodically or according to a protocol or schedule.

The fungi drying unit 106 may be based on any drying mechanism including, without limitation, thermal drying, IR drying, forced air drying, freeze drying, any other known or to be developed drying mechanism for drying fungi, or combinations thereof. For any these drying mechanism, the drying unit 106 may be configured to set the time, temperature, and pressure. The drying unit 106 may also be configured to change time, temperature, and/or pressure periodically or according to a protocol or schedule.

The fungi comminuting unit 108 may be based on any comminuting methodology including, without limitation, grinding, cryogenic-grinding, shredding, crushing, milling, titrating, pulverizing, cutting, chopping, vibrating, sonicating, sonic pulverizing, any other known comminuting methodology or to be invented comminuting methodology, or combinations thereof. For any these comminuting mechanism, the comminuting unit 106 may be configured to set the time, temperature, and pressure. The comminuting unit 106 may also be configured to change time, temperature, and/or pressure periodically or according to a protocol or schedule.

The fungi dosing preparation unit 110 may be based on any dosing preparation methodology including, without limitation, tableting, capsule filling and sealing, softgel capsule filling and sealing, tea packaging, edible supplement packaging, any other administrating format, or combinations thereof. For any these dosing preparation mechanism, the dosing preparation unit 110 may be configured to set the size, type, and number of produced administration formats. The dosing preparation unit 110 may also be configured to change size, type, and number of produced administration formats periodically or according to a protocol or schedule.

The fungi dose format packaging unit 112 may be based on any dose format packaging methodology including, without limitation, boxes, bags, glass bottles, plastic bottles, jars, any other packaging format, or combinations thereof. The dose format packaging unit 112 may also be configured to package the packaging formats in bulk containers. The dose format packaging unit 112 may also be configured to vary the dose format packages and the bulk packages periodically or according to a protocol or schedule.

### Another Embodiment

Referring now to FIG. 1B, an embodiment of an apparatus of this disclosure, generally 150, is shown to include a fungi supply unit 152, a fungi UV treating unit 154, a fungi drying unit 156, and a fungi comminuting unit 158. The apparatus 150 also includes an herb supply unit 160, an optional herb treating unit 162, an herb drying unit 164, and an herb comminuting unit 166. The apparatus 150 also includes a fungi/herb dosing preparation unit 168, which takes comminuted fungi and comminuted herbs from the fungi comminuting unit 158 and the herb comminuting unit 166, and a fungi/herb dose format packaging unit 170. The units may be include the methodologies set forth above and may be configured at set forth above.

### Specific Batch Apparatus Embodiments

One Potential Batch Apparatus Embodiment

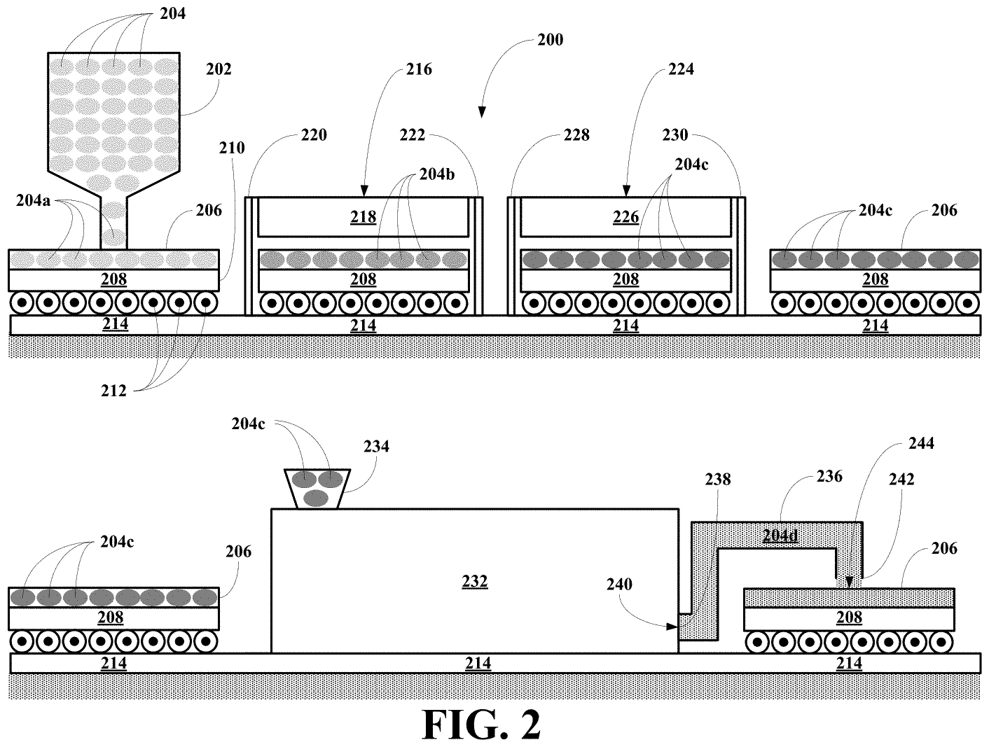

Referring now to FIG. 2, an embodiment of a batch apparatus of this disclosure, generally 200, is shown to include a fungi hopper supply unit 202 containing fungi 204 and a fungi receiving tray 206 mounted on a transport unit 208 having a tray mounting platform 210 and one or more wheel assemblies 212. The apparatus 200 also includes a track 214 upon which the transport units 208 traverse during irradiating and drying and from which their contents after drying are comminuted and the comminuted fungi are placed back in the transport units 208 for dose formatting and packaging.

The apparatus 200 also includes a UV treating unit 216 including a UV irradiation unit 218 and intensity and frequency regulators and control electronic and power supply units (not shown) that are well know in the art. The transport unit 208 enters the UV treating unit 216 through an automatic entry door 220. Once inside the UV treating unit 216, the fungi in the tray 206 is irradiated with UV light according to a UV treating protocol to form UV treated fungi 204b. After UV irradiation, the transport unit 208 leaves the UV treating unit 216 via an automatic exit door 222.

The apparatus 200 also includes a drying unit 224 including a heating element 226 and temperature sensors and regulators and control electronic and power supply units (not shown) that are well know in the art. The transport unit 208 enters the drying unit 224 through an automatic entry door 228. Once inside the drying unit 224, the fungi in the tray 206 is heated according to a drying protocol to form UV treated and dried fungi 204c. After drying, the transport unit 208 leaves the drying unit 224 via an automatic exit door 230.

The apparatus 200 also includes a comminuting unit 232 including a feeder 234. The UV treated and dried fungi 204c is fed to the feeder 234 by any known methodology and comminuted into a powdered UV treated and dried fungi 204d having a desired particle size distribution. The powdered UV treated and dried fungi 204d is then loaded onto the transport unit 208 via a conduit 236 having a proximal end 238 located near a left side bottom location 240 and a distal end 242 positioned above a center 244 of the tray 206.

The apparatus 200 also includes a control unit. The control unit includes one or more processing units including one or more processors, operating software, communication hardware and software, memory, one or more mass storage devices, one or more input devices, one or more output devices, communication pathways, one or more sensors, apparatus control routines, and other components needed to control the apparatus 200 as described more thoroughly herein.

Another Potential Batch Apparatus Embodiment

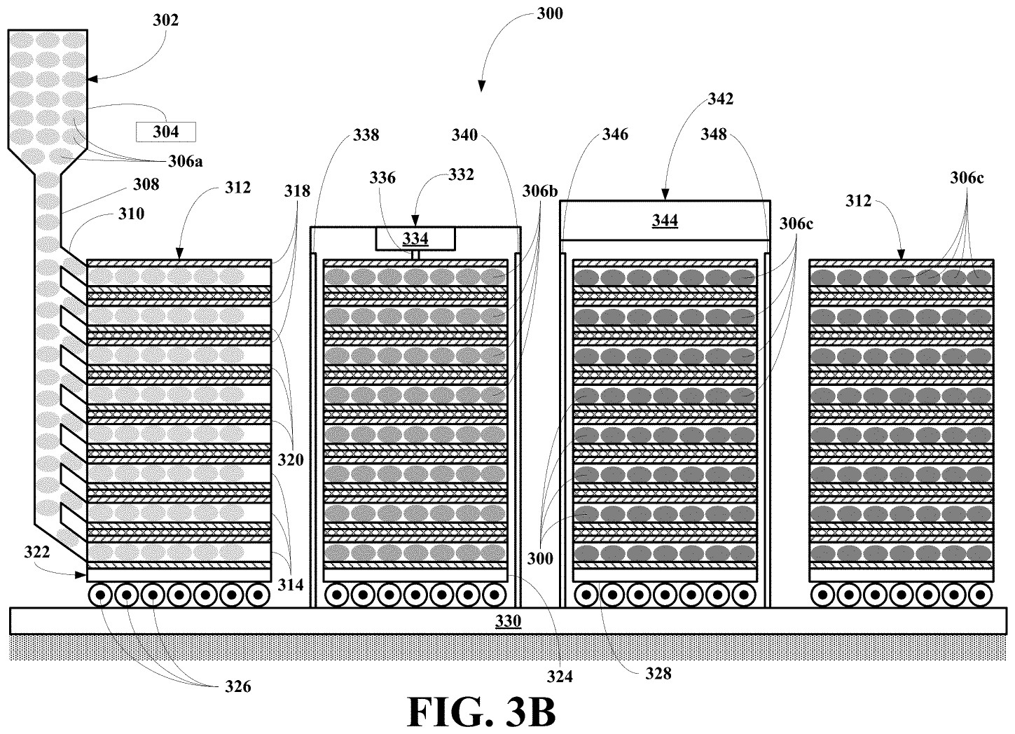

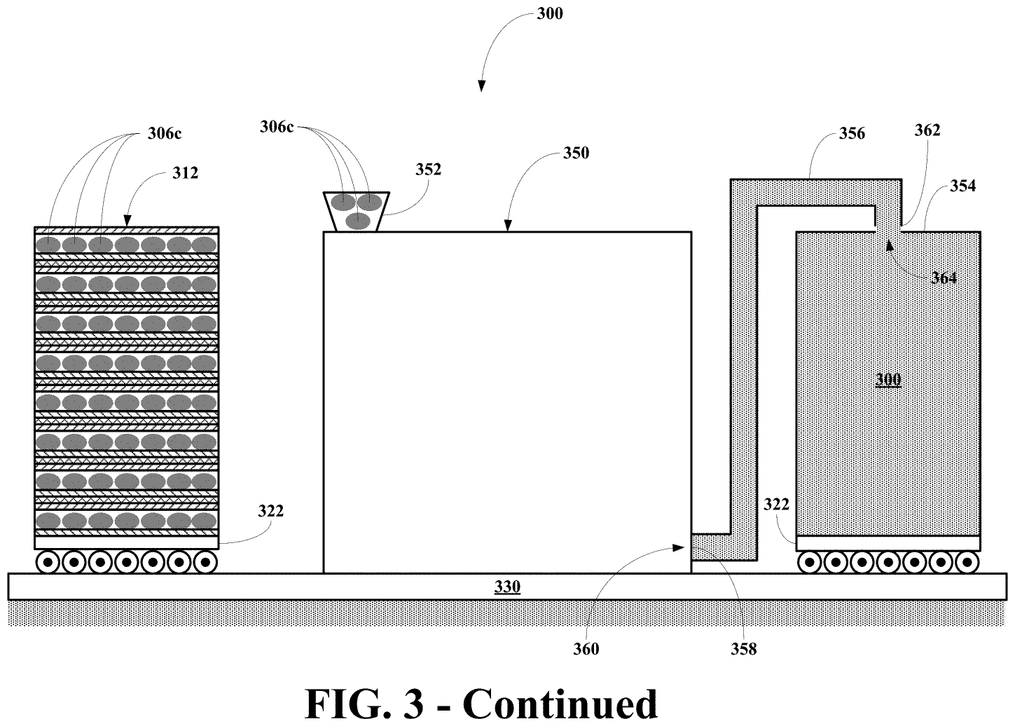

Referring now to FIG. 3, another embodiment of a batch apparatus of this disclosure, generally 300, is shown to include a fungi supply unit 302 including a hopper 304 containing raw fungi 306a. The fungi supply unit 302 also includes a supply conduit 308 including a plurality of supply slots 310.

The apparatus 300 also includes a tray assembly 312 including a plurality of UV transparent trays 314. Each of the trays 314 includes a top UV irradiating unit 318 and a bottom UV irradiating unit 320. The tray assembly 312 is mounted on a transport unit 322 including a base 324 and wheels 326 mount to a bottom 328 of the base 316. The supply slots 310 supply the raw fungi 306a to the trays 314.

The apparatus 300 also includes a track 330 upon which the transport unit 322 traverses during irradiating and drying and from which the irradiated and dried fungi are comminuted and the comminuted fungi are placed back in the transport units 322 now including a container for comminuted fungi for dose formatting and packaging as described below.

The apparatus 300 also includes a UV treating unit 332 including a UV irradiation control unit 334 including intensity and frequency regulators and control electronic and power supply units (not shown) that are well know in the art and a power conduit 336 for supplying power to the UV irradiating units 318 and 320 associated with the UV transparent trays 314. The transport unit 314 with the tray assembly 312 mounted thereon enters the UV treating unit 324 through an automatic entry door 338. Once inside the UV treating unit 332, the raw fungi 306a in the trays 314 are irradiated with UV light according to a UV treating protocol controlled by the UV irradiation control unit 336 to form UV treated fungi 306b. After UV treatment, the transport unit 322 leaves the UV treating unit 332 via an automatic exit door 340.

The apparatus 300 also includes a drying unit 342 including a heating element 344 and temperature sensors and regulators and control electronic and power supply units (not shown) that are well know in the art. The transport unit 322 enters the drying unit 342 through an automatic entry door 346. Once inside the drying unit 342, the UV treated fungi 306b in the trays 314 are heated according to a drying protocol to form UV treated and dried fungi 306c. After drying, the transport unit 322 leaves the drying unit 342 via an automatic exit door 348.

Referring now to FIG. 3 Continued, the apparatus 300 also includes a comminuting unit 350 including a feed member 352. The UV treated and dried fungi 306c is fed to the feed member 352 by any known methodology and comminuted into a powdered UV treated and dried fungi 306d having a desired particle size distribution. The powdered UV treated and dried fungi 306d is then loaded in a container 354 mounted on the transport unit 322 via a conduit 356 having a proximal end 358 located near a left side bottom location 360 and a distal end 362 positioned above a center 364 of the container 354.

The apparatus 300 also includes a control unit. The control unit includes one or more processing units including one or more processors, operating software, communication hardware and software, memory, one or more mass storage devices, one or more input devices, one or more output devices, communication pathways, one or more sensors, apparatus control routines, and other components needed to control the apparatus 300 as described more thoroughly herein.

Specific Continuous Apparatus Embodiments

### A Potential Continuous Apparatus Embodiment

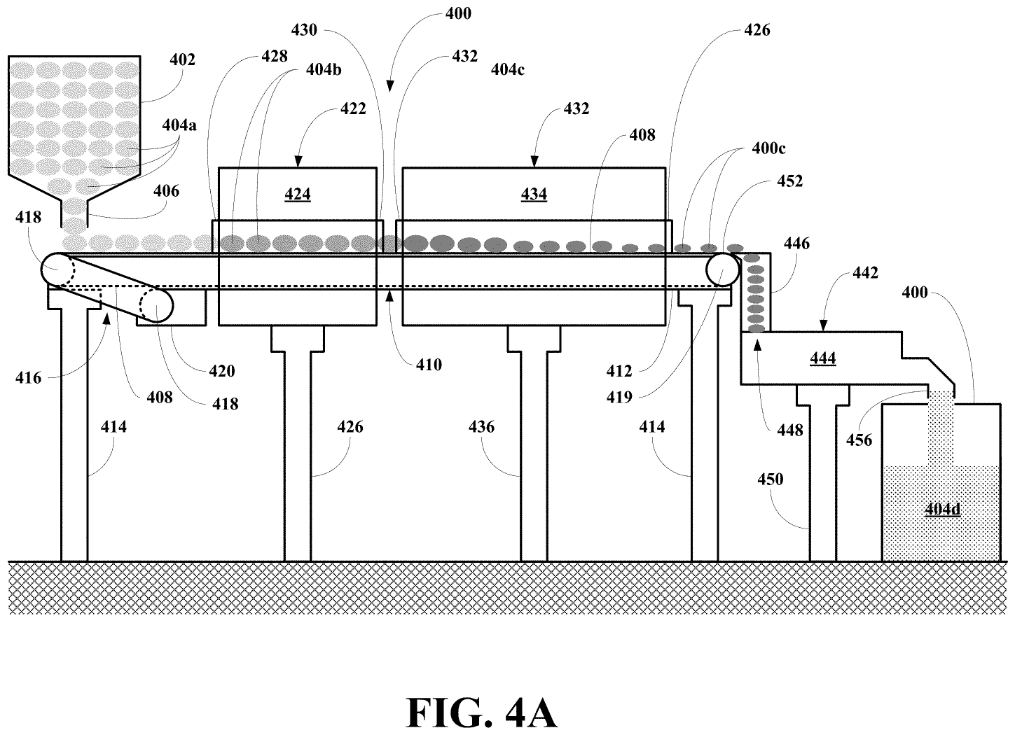

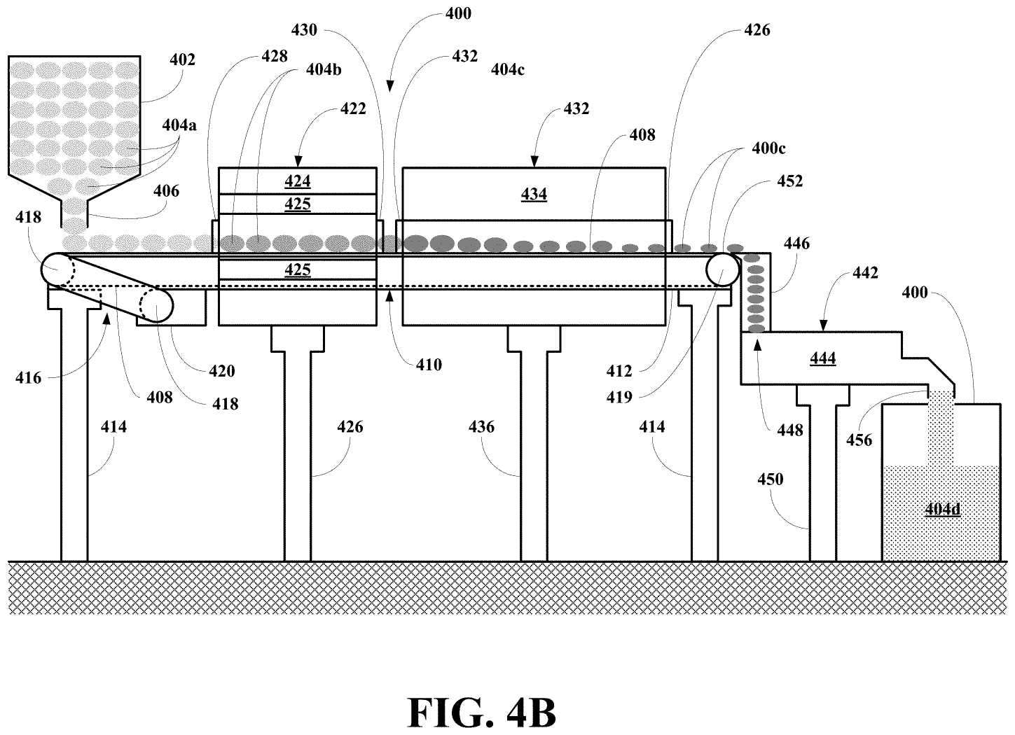

Referring now to FIG. 4A, an embodiment of a continuous apparatus of this disclosure, generally 400, is shown to include a fungi hopper supply unit 402 containing raw fungi 404a. The supply unit 402 supplies the raw fungi 404a through a supply conduit 406 onto a conveyor belt 408 of a conveyor belt unit 410. The conveyor belt unit 410 also includes a housing 412 supported by supports 414 and a drive assembly 416 including drive wheels 418, a non-drive wheel 419, a drive motor 420, and power supply (not shown).

The apparatus 400 also includes a UV treating unit 422 including a UV control and irradiation unit 424 including one or more UV output devices, intensity and frequency regulators, control electronic and power supply units (not shown) well know in the art supported by support 426. The conveyor belt 408 conveys the raw fungi 404a into the UV treating unit 422 through an automatic entry door 428. Once inside the UV treating unit 422, the raw fungi 404a are irradiated with UV light according to a UV treating protocol controlled by the UV irradiation unit 424 to form UV treated fungi 404b. After UV treatment, the UV treated fungi 404b leaves the UV treating unit 422 on the conveyor belt 408 via an automatic exit door 430. The doors 428 and 430 are designed to allow the raw fungi 404a to enter into the UV treating unit 422 with minimal UV light leakage.

The apparatus 400 also includes a drying unit 432 including a heating unit 434 and temperature sensors, regulators, control electronic and power supply units (not shown) well know in the art and is supported by support 436. The conveyor belt 408 conveys the UV treated fungi 404b into the drying unit 432 through an automatic entry door 438. Once inside the drying unit 432, the UV treated fungi 404b are heated according to a drying protocol to form UV treated and dried fungi 404c. After drying, the UV treated and dried fungi 404c leaves the drying unit 432 on the conveyor belt 408 via an automatic exit door 440. The doors 438 and 440 are designed to allow the UV treated fungi 404b into the drying unit 432 with minimal heat loss to the surrounding.

The apparatus 400 also includes a comminuting unit 442 including a comminuting device 444 having a feed conduit 446 and an inlet 448. The comminuting unit 442 also includes sensors, regulators, control electronic, and power supply units (not shown) well know in the art and is supported by support 450. The UV treated and dried fungi 404c are conveyed by the conveyor belt 408 from the drying unit 432 and fall off an end 452 of the belt 408 into the feed conduit 446 and into the comminuting device 444, where the UV treated and dried fungi 404c are comminuted to form a powdered UV treated and dried fungi 404d. The powdered UV treated and dried fungi 404d proceed into a container 454 via an outlet 456.

The apparatus 400 also includes a control unit. The control unit includes one or more processing units including one or more processors, operating software, communication hardware and software, memory, one or more mass storage devices, one or more input devices, one or more output devices, communication pathways, one or more sensors, apparatus control routines, and other components needed to control the apparatus 400 as described more thoroughly herein.

Another Potential Continuous Apparatus Embodiment

Referring now to FIG. 4B, an embodiment of a continuous apparatus of this disclosure, generally 400, is shown to include a fungi hopper supply unit 402 containing raw fungi 404a. The supply unit 402 supplies the raw fungi 404a through a supply conduit 406 onto a conveyor belt 408 of a conveyor belt unit 410. The conveyor belt unit 410 also includes a housing 412 supported by supports 414 and a drive assembly 416 including drive wheels 418, a non-drive wheel 419, a drive motor 420, and power supply (not shown).

The apparatus 400 also includes a UV treating unit 422 including a UV control unit 424 and irradiation unit 425 including one or more UV output devices, intensity and frequency regulators, control electronic and power supply units (not shown) well know in the art supported by support 426. The conveyor belt 408 conveys the raw fungi 404a into the UV treating unit 422 through an automatic entry door 428. Once inside the UV treating unit 422, the raw fungi 404a are irradiated with UV light according to a UV treating protocol controlled by the UV irradiation unit 424 to form UV treated fungi 404b. After UV treatment, the UV treated fungi 404b leaves the UV treating unit 422 on the conveyor belt 408 via an automatic exit door 430. The doors 428 and 430 are designed to allow the raw fungi 404a to enter into the UV treating unit 422 with minimal UV light leakage.

The apparatus 400 also includes a drying unit 432 including a heating unit 434 and temperature sensors, regulators, control electronic and power supply units (not shown) well know in the art and is supported by support 436. The conveyor belt 408 conveys the UV treated fungi 404b into the drying unit 432 through an automatic entry door 438. Once inside the drying unit 432, the UV treated fungi 404b are heated according to a drying protocol to form UV treated and dried fungi 404c. After drying, the UV treated and dried fungi 404c leaves the drying unit 432 on the conveyor belt 408 via an automatic exit door 440. The doors 438 and 440 are designed to allow the UV treated fungi 404b into the drying unit 432 with minimal heat loss to the surrounding.

The apparatus 400 also includes a comminuting unit 442 including a comminuting device 444 having a feed conduit 446 and an inlet 448. The comminuting unit 442 also includes sensors, regulators, control electronic, and power supply units (not shown) well know in the art and is supported by support 450. The UV treated and dried fungi 404c are conveyed by the conveyor belt 408 from the drying unit 432 and fall off an end 452 of the belt 408 into the feed conduit 446 and into the comminuting device 444, where the UV treated and dried fungi 404c are comminuted to form a powdered UV treated and dried fungi 404d. The powdered UV treated and dried fungi 404d proceed into a container 454 via an outlet 456.

The apparatus 400 also includes a control unit. The control unit includes one or more processing units including one or more processors, operating software, communication hardware and software, memory, one or more mass storage devices, one or more input devices, one or more output devices, communication pathways, one or more sensors, apparatus control routines, and other components needed to control the apparatus 400 as described more thoroughly herein.

### Embodiments of Control and Power Systems for the Apparatuses

### An Embodiment

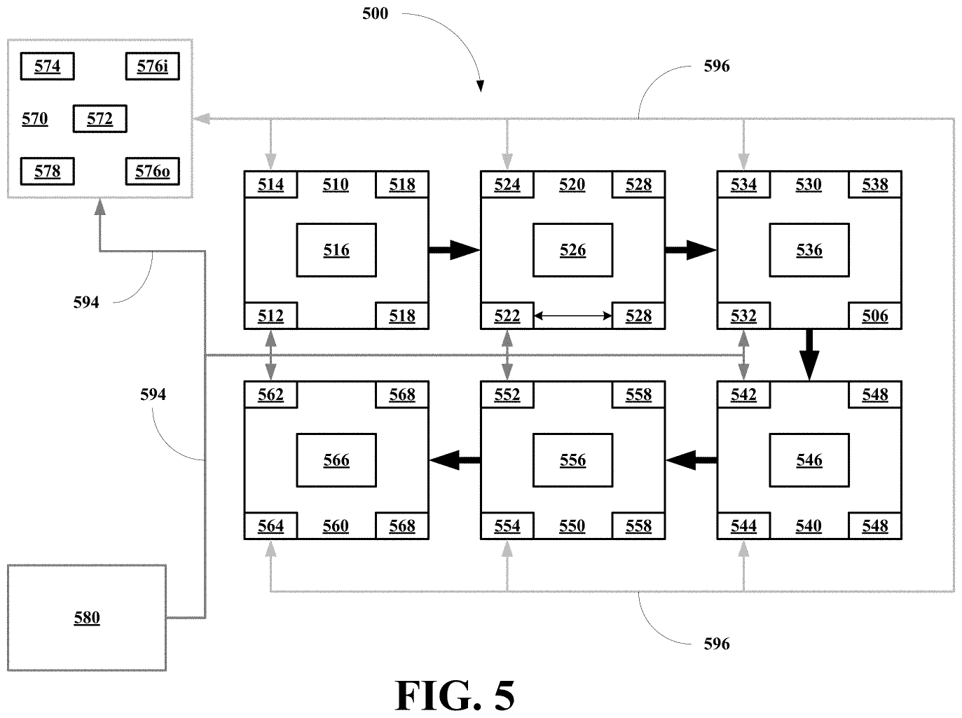

Referring now to FIG. 5, an embodiment of a control and monitoring assembly associated with the apparatuses of this disclosure, generally 500, is shown.

The apparatus 500 also includes a fungi supply unit 510 including a supply unit power supply 512, a supply unit controller 514, a supply unit feeder 516, and supply unit sensors 518. The power supply 512 supplies power to the controller 514, the feeder 516, and the sensors 518 via power supply pathways (not shown) and the controller 514 receives information and controls the feeder 516 and the sensors 518 via bilateral communication pathways (not shown).

The apparatus 500 also includes a fungi UV treating unit 520 including a UV treating unit power supply 522, a UV treating unit controller 524, a UV treating unit UV irradiating device 526, and UV treating unit sensors 528. The power supply 522 supplies power to the controller 524, the device 526, and the sensors 528 via power supply pathways (not shown) and the controller 524 receives information and controls the feeder 526 and the sensors 528 via bilateral communication pathways (not shown).

The apparatus 500 also includes a fungi drying unit 530 including a drying unit power supply 532, a drying unit controller 534, a drying unit heater 536, and drying unit sensors 538. The power supply 532 supplies power to the controller 534, the heater 536, and the sensors 538 via power supply pathways (not shown) and the controller 534 receives information and controls the heater 536 and the sensors 538 via bilateral communication pathways (not shown).

The apparatus 500 also includes a fungi comminuting unit 540 including a comminuting unit power supply 542, a comminuting unit controller 544, a comminuting unit comminutor 546, and comminuting unit sensors 548. The power supply 542 supplies power to the controller 544, the comminutor 546, and the sensors 548 via power supply pathways (not shown) and the controller 544 receives information and controls the comminutor 546 and the sensors 548 via bilateral communication pathways (not shown).

The apparatus 500 also includes a fungi dosing format preparation unit 550 including a dosing format preparation unit power supply 552, a dosing format preparation unit controller 554, a dosing format preparation filler 556, and dosing preparation unit sensors 558. The power supply 552 supplies power to the controller 554, the filler 546, and the sensors 558 via power supply pathways (not shown) and the controller 554 receives information and controls the filler 556 and the sensors 558 via bilateral communication pathways (not shown).

The apparatus 500 also includes a fungi dose format packaging unit 560 including a dose format packaging unit power supply 562, a dose format packaging unit controller 564, a dose format packaging unit packer 566, and dose format packaging unit sensors 568. The power supply 562 supplies power to the controller 564, the packer 566, and the sensors 568 via power supply pathways (not shown) and the controller 564 receives information and controls the packer 566 and the sensors 568 via bilateral communication pathways (not shown).

The apparatus 500 also includes a control unit 570 including a processing unit 572 and memory devices and mass storage devices 574, one or more input devices 576i, one or more output devices 576o, and one or more communication devices 578. The power supply 572 supplies power to the devices 574, the devices 576i&o, and the devices 568 via power supply pathways (not shown) and the processing unit 562 receives information and controls the devices 574, the devices 566i&o and the devices 568 via bilateral communication pathways (not shown). The control unit 570 is in bilateral communication with the unit controllers 514, 524, 534, 544, 554, and 564 via communication pathways 590.

It should be recognized that the unit controllers 514, 524, 534, 544, 554, and 564 may include all or some of the components of the control unit 570 including memory device and mass storage devices, one or more input devices, one or more output devices, and one or more communication devices.

The apparatus 500 also includes a power supply unit 580. The power supply unit 580 supplies power to the unit power supplies 512, 522, 532, 542, 552, and 562, and the control unit 580 via power conducting pathways 592.

### Another Embodiment

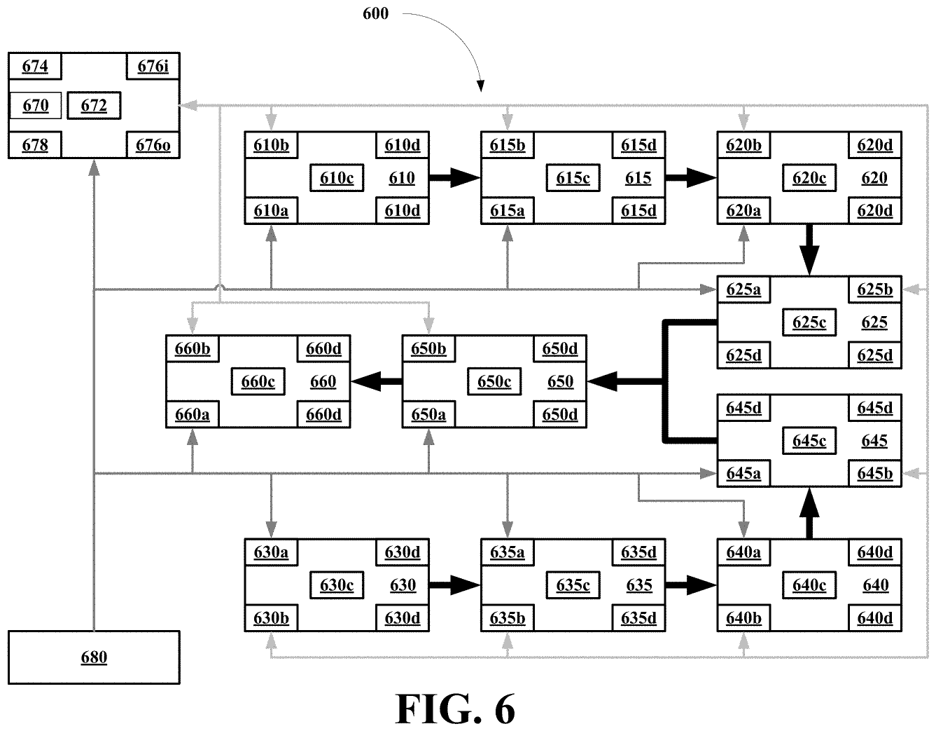

Referring now to FIG. 6, an embodiment of a control and monitoring assembly associated with the apparatuses of this disclosure, generally 600, is shown.

Fungi Line

The apparatus 600 also includes a fungi supply unit 610 including a supply unit power supply 610a, a supply unit controller 610b, a supply unit feeder 610c, and supply unit sensors 610d. The power supply 610a supplies power to the controller 610b, the feeder 610c, and the sensors 610d via power supply pathways (not shown) and the controller 610b receives information and controls the feeder 610c and the sensors 610c via bilateral communication pathways (not shown).

The apparatus 600 also includes a fungi UV treating unit 615 including a UV treating unit power supply 615a, a UV treating unit controller 615b, a UV treating unit UV irradiating device 615c, and UV treating unit sensors 615d. The power supply 615a supplies power to the controller 615b, the device 615c, and the sensors 615d via power supply pathways (not shown) and the controller 615b receives information and controls the feeder 615c and the sensors 615d via bilateral communication pathways (not shown).

The apparatus 600 also includes a fungi drying unit 620 including a drying unit power supply 620a, a drying unit controller 620b, a drying unit heater 620c, and drying unit sensors 620d. The power supply 620a supplies power to the controller 620b, the heater 620c, and the sensors 620d via power supply pathways (not shown) and the controller 620b receives information and controls the heater 620c and the sensors 620d via bilateral communication pathways (not shown).

The apparatus 600 also includes a fungi comminuting unit 625 including a comminuting unit power supply 625a, a comminuting unit controller 625b, a comminuting unit comminutor 625c, and comminuting unit sensors 625d. The power supply 625a supplies power to the controller 625b, the comminutor 625c, and the sensors 625d via power supply pathways (not shown) and the controller 625b receives information and controls the comminutor 625c and the sensors 625d via bilateral communication pathways (not shown).

Herb Line

The apparatus 600 also includes a herb supply unit 630 including a supply unit power supply 630a, a supply unit controller 630b, a supply unit feeder 630c, and supply unit sensors 630d. The power supply 630a supplies power to the controller 630b, the feeder 630c, and the sensors 630d via power supply pathways (not shown) and the controller 630b receives information and controls the feeder 630c and the sensors 630c via bilateral communication pathways (not shown).

The apparatus 600 also includes an optional herb treating unit 635 including a treating unit power supply 635a, a treating unit controller 635b, a treating unit irradiating device 635c, and treating unit sensors 635d. The power supply 635a supplies power to the controller 635b, the device 635c, and the sensors 635d via power supply pathways (not shown) and the controller 635b receives information and controls the feeder 635c and the sensors 635d via bilateral communication pathways (not shown).

The apparatus 600 also includes a herb drying unit 640 including a drying unit power supply 640a, a drying unit controller 640b, a drying unit heater 640c, and drying unit sensors 640d. The power supply 640a supplies power to the controller 640b, the heater 640c, and the sensors 640d via power supply pathways (not shown) and the controller 640b receives information and controls the heater 640c and the sensors 640d via bilateral communication pathways (not shown).

The apparatus 600 also includes a herb comminuting unit 645 including a comminuting unit power supply 645a, a comminuting unit controller 645b, a comminuting unit comminutor 645c, and comminuting unit sensors 645d. The power supply 645a supplies power to the controller 645b, the comminutor 645c, and the sensors 645d via power supply pathways (not shown) and the controller 645b receives information and controls the comminutor 645c and the sensors 645d via bilateral communication pathways (not shown).

Dosing and Packaging

The apparatus 600 also includes a dosing format preparation unit 650 including a dosing format preparation unit power supply 650a, a dosing format preparation unit controller 650b, a dosing format preparation filler 650c, and dosing preparation unit sensors 650d. The power supply 662a supplies power to the controller 664b, the filler 646c, and the sensors 668d via power supply pathways (not shown) and the controller 664b receives information and controls the filler 666c and the sensors 668d via bilateral communication pathways (not shown).

The apparatus 600 also includes a dose format packaging unit 660 including a dose format packaging unit power supply 660a, a dose format packaging unit controller 660b, a dose format packaging unit packer 660c, and dose format packaging unit sensors 660d. The power supply 660a supplies power to the controller 660b, the packer 660c, and the sensors 660d via power supply pathways (not shown) and the controller 660b receives information and controls the packer 660c and the sensors 660d via bilateral communication pathways (not shown).

Power and Control

The apparatus 600 also includes a control unit 670 including a processing unit 672 and memory devices and mass storage devices 674, one or more input devices 676i, one or more output devices 676o, and one or more communication devices 678. The power supply 672 supplies power to the devices 674, the devices 676i&o, and the devices 678 via power supply pathways (not shown) and the processing unit 662 receives information and controls the devices 674, the devices 666i&o and the devices 668 via bilateral communication pathways (not shown). The control unit 670 is in bilateral communication with the unit controllers 610b, 615b, 620b, 625b, 630b, 635b, 640b, 645b, 650b, and 660b via communication pathways 690.

It should be recognized that the unit controllers 610b, 615b, 620b, 625b, 630b, 635b, 640b, 645b, 650b, and 660b may include all or some of the components of the control unit 670 including memory device and mass storage devices, one or more input devices, one or more output devices, and one or more communication devices.

The apparatus 600 also includes a power supply unit 680. The power supply unit 680 supplies power to the unit power supplies 610a, 615a, 620a, 625a, 630a, 635a, 640a, 645a, 650a, and 660a, and the control unit 680 via power conducting pathways 692.

### Embodiments of Methods

Fungi Compositions

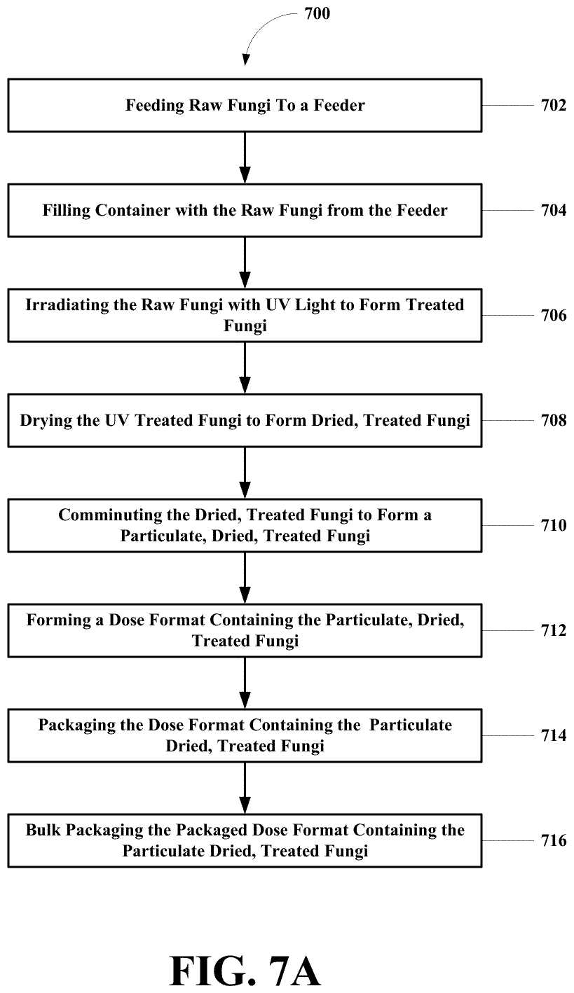

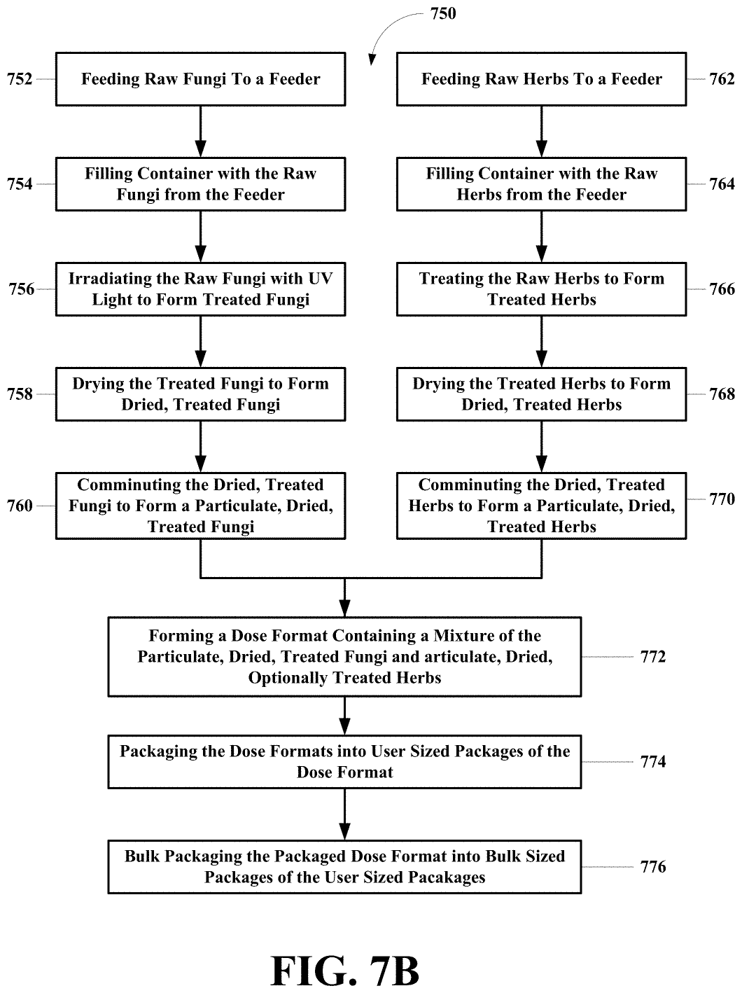

Referring now to FIG. 7A, an embodiment of a method for making the fungi compositions of this disclosure, generally 700, is shown to include feeding raw fungi to a feeder in a feeding step 702. The method 700 also includes the step of filling one or more containers with the raw fungi in a filling step 704. The method 700 also includes irradiating the raw fungi in the container with UV light in an irradiating step 706 to form treated fungi. The method 700 also includes drying the treated fungi in a drying step 708 to form dried, treated fungi. The method 700 also includes comminuting the dried, treated fungi in a comminuting step 710 to form particulate, dried, treated fungi. The method 700 also includes forming a dose format of the particulate, dried, treated fungi in a dose forming step 712 to form a dose form containing the particulate, dried, treated fungi. The method 700 also includes packaging the dose form in a packaging step 714 to form user sized packages of the dose formats. The method 700 also includes bulk packaging the user packages in a bulk packaging step 716 to form bulk package sized packages of the user packages.

Mixed Fungi and Herb Compositions

Referring now to FIG. 7B, another embodiment an embodiment of a method for making the fungus and herb compositions of this disclosure, generally 750, is shown.

Fungi Steps

The method 750 includes feeding raw fungi to a feeder in a feeding step 752. The method 750 also includes the step of filling one or more containers with the raw fungi in a filling step 754. The method 750 also includes irradiating the raw fungi in the container with UV light in an irradiating step 756 to form treated fungi. The method 750 also includes drying the treated fungi in a drying step 758 to form dried, treated fungi. The method 750 also includes comminuting the dried, treated fungi in a comminuting step 760 to form particulate, dried, treated fungi.

Herb Steps

The method 750 includes feeding a raw herbs to a feeder in a feeding step 762. The method 750 also includes the step of filling one or more containers with the raw herbs in a filling step 764. The method 750 also includes optionally treating the raw herbs in the container in a treating step 766 to form optionally treated herbs. The method 750 also includes drying the optionally treated herbs in a drying step 768 to form dried, optionally treated herbs. The method 750 also includes comminuting the dried, optionally treated herbs in a comminuting step 770 to form particulate, dried, optionally treated herbs.

Dose Formating and Packaging Steps

The method 750 also includes forming a dose format of a mixture of the particulate, dried, treated fungi and the particulate, dried, optionally treated herbs in a dose forming step 772 to form a dose form containing the mixture. The method 750 also includes packaging the dose form in a packaging step 774 to form packages of the dose form in user sized packages of the dose formats. The method 750 also includes bulk packaging the user packages in a bulk packaging step 776 to form bulk package sized of the user packages.

### Closing Paragraph of the Disclosure

All references cited herein are incorporated by reference. Although the disclosure has been disclosed with reference to its preferred embodiments, from reading this description those of skill in the art may appreciate changes and modification that may be made which do not depart from the scope and spirit of the disclosure as described above and claimed hereafter.

We claim:

1. A composition comprising:
a first effective amount of one or more powdered, dried, ultraviolet (UV) treated fungus materials, wherein the effective amount is sufficient to deliver between about 500 international unit (IU) and about 4000 IU of vitamin D2 per day to a patient.

2. The composition of claim 1, wherein the first effective amount is between about 750 IU and 3500 IU of vitamin D2 per day, between about 1000 IU and 3500 IU of vitamin D2 per day, between about 1000 IU and 3500 IU of vitamin D2 per day, between about 1500 IU and 3500 IU of vitamin D2 per day, between about 1750 IU and 3500 IU of vitamin D2 per day, or between about 2000 IU and 3500 IU of vitamin D2 per day.

3. The composition of claim 1, wherein the one or more fungus materials comprises Cordyceps sinensis; Ganoderma lucidum; G. tsugae; reishi; Lentinula edodes; Hericium erinaceus; Gametes versicolor; Inonotus obliquus; Ganoderma lucidum; Grifola frondosa; Lentinula edodes; Agaricus blazei; Wolfiporia extensa; fit ling; Tremella fuciformis; Laricifomes officinalis; Phellinus linteus; Boletus frondosus; Cordyceps spp.; Fomes fomentarius; Ganoderma lucidum; Grifola frondosa; Hericium erinaceus; Inonotus obliquus; Ophiocordyceps sinensis; phellinus spp.; Polyporus umbellatus; Taiwanofungus camphoratus; Wolfiporia extensa; Entoloma abortivum; Ganoderma applanatum; Fistulina hepatica; Craterellus cornucopioides; C. cenerius; C. foetidus; Eutypella scoparia; cantharellus; Laetiporus sulphureus; Ustilago maydis; Cordyceps; Cordyceps militaris; cremini; Hypsizygus ulmarius; enoki/enokitake; Peniophora albobadia; Thelephora gambajun; Calvatia gigantea; chlorophyllum; Sarcodon imbricatus; Hydnum repandum; Hydnum umbilicatum; Craterellus cornucopiodes; Hericium erinaceus; Hypomyces lactifluorum; Grifola frondosa; Agaricus; morchella; Pleurotus ostreatus; Gomphus clavatus; Boletus edulis; Ganoderma lucidum; coprinus; shimeji; Lentinula edodes; Suillus boletus; lepiota; Tremella fuciformis; Volvariella volvacea; laetiporus; Clavariadelphus cornucopiodes; termitomyces; Agaricus bisporus; pleurotus species; Lentinula edodes; Auricularia auricula-judae; volvariella volvacea; Flammulina velutipes; Tremella fuciformis; Hypsizygus tessellatus; Hypsizygus marmoreus; bunapi-shimeji; buna-shimeji; Stropharia rugosoannulata; Cyclocybe aegerita; Hericium erinaceus; Boletus edulis; Calbovista subsculpta; Calvatia gigantea; Cantharellus cibarius; Craterellus tubaeformis; Clitocybe nuda; Cortinarius caperatus; Craterellus cornucopioides; Grifola frondosa; Gyromitra esculenta; Hericium erinaceus; Hydnum repandum; Lactarius deliciosus; Morchella conica var deliciosa; Morchella esculenta var.; Rotunda; pleurotus species; Tricholoma matsutake; Tuber aestivum; Tuber borchii; Tuber brumale; Tuber indicum; Tuber macrosporum; Tuber mesentericum; Agaricus arvensis; Agaricus silvaticus; Amanita caesarea; Armillaria mellea; Boletus Badius; Calocybe gambosa; Calvatia utriformis; Lycoperdon caelatum; Chroogomphus species; clavariaceae species; clavulinaceae species; Coprinus comatus; Cortinarius variicolor; Cyttaria espinosae; Fistulina hepatica; Flammulina velutipes; Hygrophorus chrysodon; Kalaharituber pfeilii; Auricularia auricula-judae; Lactarius deterrimus; Lactarius salmonicolor; Lactarius salmonicolor; Lactarius subdulcis; Lactarius volemus; Laetiporus sulphureus; Leccinum aurantiacum; Leccinum scabrum; Leccinum versipelle; Macrolepiota procera; Marasmius oreades; Polyporus mylittae; Polyporus squamosus; Ramariaceae species; Rhizopogon luteolus; Russula laeta; Sparassis crispa; Suillus bovinus; Suillus granulatus; Suillus luteus; Suillus tomentosus; Tricholoma terreum; Amanita fulva; Amanita rubescens; Coprinopsis atramentaria; Gyromitra esculenta; lactarius spp.; Lepista saeva; Verpa bohemica; or mixtures and combinations thereof.

4. The composition of claim 1, further comprising:
a second effective amount of powdered, dried one or more herb materials, wherein the second effective amount is between about 0.1 gand about 5 g per day.

5. The composition of claim 4, wherein the second effective amount is between about 0.1 g and about 4 g, between about 0.1 g and about 3 g, between about 0.2 g and about 4 g, between about 0.2 g and about 3 g, between about 0.5 g and about 4 g, or between about 0.5 g and about 3 g.

6. The composition of claim 4, wherein the one or more herb materials comprise Prunella vulgaris, Artemisi, annua, turmeric, torreyanucifera, Ocimum basilicum, Canistrocarpus cervicornis, Padina gymnospora, Palisada perforate, Caulerpa racemose, Terminalia chebula, Raoulia australis, Woodforia fruticose, Cordydalis saxicola, Silybum marianum, Agrimonia eupatoria; Medicago sativa; Aloe vera; Panax quinquefolius; Phyllanthus emblica; Angelica archangelica; Pimpinella anisum; Arnica montana; Withania somnifera; Astragalus membranaceus; Eleutherococcus senticosus; Bacopa monnieri; Arctostaphylos uva-ursi; Monarda didyma; Entomophile pollen; Vaccinium mytillus; Prunus serotina; Cimicifuga racemosa; Eupatorium perfoliatum; Borago officinalis; boswellia serrata; Agathosma betulina; Arctium lappa; Petasites hybridus; Calendula officinalis; Frangula purshiana; Nepeta cataria; Uncaria tomentosa; Capsicum annuum; Matricaria recutita; Larrea tridentata; Vitex agnus-castus; Cichorium intybus; Innamonum verum; Lycopodium clavatum; Symphytum officinale; Cordyceps sinensis; Taraxacum officinale; Angelica sinensis; Echinacea purpurea; Polygonum multiflorum; Ginkgo bilobar centella asictica; Gynostemma pentaphyllum; Ocimum sanctum; Piper methysticum; Panax ginseng; Cymbopogon citratus; Glycyrrhiza uralensis; Hericium erinaceus; Lycium barbarum; Lepidium meyenii; Silybum marianum; Grifola frondosa; Rhodiola rosea; Serenoa repens; Schizandra chinensis; Asphaltum; Eleutherococcus senticosus; Scutellaria lateriflora; Hypericum perforatum; Pfaffia paniculata; Curcuma longa; Valerian officinalis; Ligustrum lucidum; Ocimum tenuiflorum syn; Ocimum sanctum; Panax quinquefolius; Rhodiola rosea; Sambucus canadensis; S. nigra; Schisandra chinensis; Wu wei zi; Withania somnifera; ashwagandha; or mixtures and combinations thereof.

7. The composition of claim 4, further comprising:
a powdered, dried, UV treated fungus material including:
about 1 g per day of ganoderma spp.,
about 1.5 g per day of Cordyceps militaris,
about 1.5 g per day of Ophiocordyceps sinensis,
about 1 g per day of Trametes versicolor,
about 1 g per day of Lentinula edodes, or
mixtures thereof,
a powdered dried herb material including:
about 3 g per day of Astragalus membranaceus,
about 1 g per day of Codonopsis pilosula,
about 1 g per day of Glycyrrhiza glabra,
about 1 g per day of Ocimum tenuiflorum,
about 1 g per day of Ocimum basilicum,
about 1 g per day of Prunella vulgaris,
about 1 g per day of Liquidambar spp.,
about 1 g per day of Origanum vulgare,
about 1 g per day of sage,
about 1 g per day of Uncaria tomentosa,
about 1 g per day of Foeniculum vulgare,
between about 1 g and about 5 g per day of Allium sativum ,
about 1.5 g per day of Prostanthera melissifolia,
between about 0.5 g and about 2 g per day of Salvia rosmarinus,
between about 0.5 g and about 2 g per day of Zingiber officinale,
between about 0.4 and about 1 g per day of echinacea spp.,
between about 1 g and about 3 g per day of Sambucus nigra,
between about 1 g and about 3 g per day of ginseng,
between about 0.5 g and about 2 g per day of Acmella oleracea,
between about 1 g and about 3 g per day of Atractylodes ovata,
between about 0.5 g and about 2 g per day of Angelica sinensis,
between about 0.5 g and about 2 g per day of Ligustrum lucidum,
between about 0.9 g and about 2 g per day of Vitamin C,
between about 1 g and about 3 g per day rhodiola,
between about 0.1 gand about 1 g per day about of Silybum marianum, or
mixtures and combinations thereof.

8. The composition of claim 1, wherein the UV treatment includes irradiating one or more raw fungus materials with UV light having a frequency or a frequency range of UV-A light, UV-B light, UV-C light or combinations thereof, at a temperature between about 20° C. and 40° C., at a moisture content between about 50% and about 90%, and for a period of time between 30 minutes and 12 hours.

9. A composition comprising:
a fungus effective amount of one or more powdered, dried, ultraviolet (UV) treated fungus materials, wherein the fungus effective amount is sufficient to deliver between about 500 international unit (IU) and about 4000 IU of vitamin D2 per day to a patient, and
a herb effective amount of powdered, dried one or more herb materials, wherein the herb effective amount is between about 0.1 gand about 5 g per day.

10. The composition of claim 9, wherein the first effective amount is between about 750 IU and 3500 IU of vitamin D2 per day, between about 1000 IU and 3500 IU of vitamin D2 per day, between about 1000 IU and 3500 IU of vitamin D2 per day, between about 1500 IU and 3500 IU of vitamin D2 per day, between about 1750 IU and 3500 IU of vitamin D2 per day, or between about 2000 IU and 3500 IU of vitamin D2 per day.

11. The composition of claim 9, wherein the one or more fungus materials comprises Cordyceps sinensis; Ganoderma lucidum; G. tsugae; reishi; Lentinula edodes; Hericium erinaceus; Gametes versicolor; Inonotus obliquus; Ganoderma lucidum; Grifola frondosa; Lentinula edodes; Agaricus blazei; Wolfiporia extensa; fú ling; Tremella fuciformis; Laricifomes officinalis; Phellinus linteus; Boletus frondosus; Cordyceps spp.; Fomes fomentarius; Ganoderma lucidum; Grifola frondosa; Hericium erinaceus; Inonotus obliquus; Ophiocordyceps sinensis; phellinus spp.; Polyporus umbellatus; Taiwanofungus camphoratus; Wolfiporia extensa; Entoloma abortivum; Ganoderma applanatum; Fistulina hepatica; Craterellus cornucopioides; C. cenerius; C. foetidus; Eutypella scoparia; cantharellus; Laetiporus sulphureus; Ustilago maydis; Cordyceps; Cordyceps militaris; cremini; Hypsizygus ulmarius; enoki/enokitake; Peniophora albobadia; Thelephora gambajun; Calvatia gigantea; chlorophyllum; Sarcodon imbricatus; Hydnum repandum; Hydnum umbilicatum; Craterellus cornucopiodes; Hericium erinaceus; Hypomyces lactifluorum; Grifola frondosa; Agaricus; morchella; Pleurotus ostreatus; Gomphus clavatus; Boletus edulis; Ganoderma lucidum; coprinus; shimeji; Lentinula edodes; Suillus boletus; lepiota; Tremella fuciformis; Volvariella volvacea; laetiporus; Clavariadelphus cornucopiodes; termitomyces; Agaricus bisporus; pleurotus species; Lentinula edodes; Auricularia auricula-judae; Volvariella volvacea; Flammulina velutipes; Tremella fuciformis; Hypsizygus tessellatus; Hypsizygus marmoreus; bunapi-shimeji; buna-shimeji; Stropharia rugosoannulata; Cyclocybe aegerita; Hericium erinaceus; Boletus edulis; Calbovista subsculpta; Calvatia gigantea; Cantharellus cibarius; Craterellus tubaeformis; Clitocybe nuda; Cortinarius caperatus; Craterellus cornucopioides; Grifola frondosa; Gyromitra esculenta; Hericium erinaceus; Hydnum repandum; Lactarius deliciosus; Morchella conica var deliciosa; Morchella esculenta var.; Rotunda; pleurotus species; Tricholoma matsutake; Tuber aestivum; Tuber borchii; Tuber brumale; Tuber indicum; Tuber macrosporum; Tuber mesentericum; Agaricus arvensis; Agaricus silvaticus; Amanita caesarea; Armillaria mellea; Boletus badius; Calocybe gambosa; Calvatia utriformis; Lycoperdon caelatum; chroogomphus species; clavariaceae species; clavulinaceae species; Coprinus comatus; Cortinarius variicolor; Cyttaria espinosae; Fistulina hepatica; Flammulina velutipes; Hygrophorus chrysodon; Kalaharituber pfeilii; Auricularia auricula-judae; Lactarius deterrimus; Lactarius salmonicolor; Lactarius salmonicolor; Lactarius subdulcis; Lactarius volemus; Laetiporus sulphureus; Leccinum aurantiacum; Leccinum scabrum; Leccinum versipelle; Macrolepiota procera; Marasmius oreades; Polyporus mylittae; Polyporus squamosus; ramariaceae species; Rhizopogon luteolus; Russula laeta; Sparassis crispa; Suillus bovinus; Suillus granulatus; Suillus luteus; Suillus tomentosus; Tricholoma terreum; Amanita fulva; Amanita rubescens; Coprinopsis atramentaria; Gyromitra esculenta; lactarius spp.; Lepista saeva; Verpa bohemica; or mixtures and combinations thereof.

12. The composition of claim 9, wherein the second effective amount is between about 0.1 g and about 4 g, between about 0.1 g and about 3 g, between about 0.2 g and about 4 g, between about 0.2 g and about 3 g, between about 0.5 g and about 4 g, or between about 0.5 g and about 3 g.

13. The composition of claim 9, wherein the one or more herb materials comprise Prunella vulgaris, Artemisi, annua, turmeric, Torreya nucifera, Ocimum basilicum, Canistrocarpus cervicornis, Padina gymnospora, Palisada perforate, Caulerpa racemose, Terminalia chebula, Raoulia australis, Woodforia fruticose, Cordydalis saxicola, Silybum marianum, Agrimonia eupatoria; Medicago sativa; Aloe vera; Panax quinquefolius; Phyllanthus emblica; Angelica archangelica; Pimpinella anisum; Arnica montana; Withania somnifera; Astragalus membranaceus; Eleutherococcus senticosus; Bacopa monnieri; Arctostaphylos uva-ursi; Monarda didyma; Entomophile pollen; Vaccinium mytillus; Prunus serotina; Aimicifuga racemosa; Eupatorium perfoliatum; Borago officinalis; Boswellia serrata; Agathosma betulina; Arctium lappa; Petasites hybridus; Calendula officinalis; Frangula purshiana; Nepeta cataria; Uncaria tomentosa; Capsicum annuum; Matricaria recutita; Larrea tridentata; Vitex agnus-castus; Cichorium intybus; Innamonum verum; Lycopodium clavatum; Symphytum officinale; Cordyceps sinensis; Taraxacum officinale; Angelica sinensis; Echinacea purpurea; Polygonum multiflorum; Ginkgo bilobar centella asictica; Gynostemma pentaphyllum; Ocimum sanctum; Piper methysticum; Panax ginseng; Cymbopogon citratus; Glycyrrhiza uralensis; Hericium erinaceus; Lycium barbarum; Lepidium meyenii; Silybum marianum; Grifola frondosa; Rhodiola rosea; Serenoa repens; Schizandra chinensis; asphaltum; Eleutherococcus senticosus; Scutellaria lateriflora; Hypericum perforatum; Pfaffia paniculata; Curcuma longa; Valerian officinalis; Ligustrum lucidum; Ocimum tenuiflorum syn; Ocimum sanctum; Panax quinquefolius; Rhodiola rosea; Sambucus canadensis; S. nigra; Schisandra chinensis; Wu wei zi; Withania somnifera; ashwagandha; or mixtures and combinations thereof.

14. The composition of claim 9, further comprising:
a powdered, dried, UV treated fungus material including:
about 1 g per day of ganoderma spp.,
about 1.5 g per day of Cordyceps militaris,
about 1.5 g per day of Ophiocordyceps sinensis,
about 1 g per day of Trametes versicolor,
about 1 g per day of Lentinula edodes, or
mixtures thereof,
a powdered dried herb material including:
about 3 g per day of Astragalus membranaceus,
about 1 g per day of Codonopsis pilosula,
about 1 g per day of Glycyrrhiza glabra,
about 1 g per day of Ocimum tenuiflorum,
about 1 g per day of Ocimum basilicum,
about 1 g per day of Prunella vulgaris,
about 1 g per day of Liquidambar spp.,
about 1 g per day of Origanum vulgare,
about 1 g per day of sage,
about 1 g per day of Uncaria tomentosa,
about 1 g per day of Foeniculum vulgare,
between about 1 g and about 5 g per day of Allium sativum ,
about 1.5 g per day of Prostanthera melissifolia,
between about 0.5 g and about 2 g per day of Salvia rosmarinus,
between about 0.5 g and about 2 g per day of Zingiber officinale,
between about 0.4 and about 1 g per day of echinacea spp.,
between about 1 g and about 3 g per day of Sambucus nigra,
between about 1 g and about 3 g per day of ginseng, between about 0.5 g and about 2 g per day of Acmella oleracea, between about 1 g and about 3 g per day of Atractylodes ovata, between about 0.5 g and about 2 g per day of Angelica sinensis, between about 0.5 g and about 2 g per day of Ligustrum lucidum, between about 0.9g and about 2 g per day of Vitamin C, between about 1 g and about 3 g per day rhodiola, between about 0.1 gand about 1 g per day about of Silybum marianum, or mixtures and combinations thereof.

15. The composition of claim 9, wherein the UV treatment includes irradiating one or more raw fungus materials with UV light having a frequency or a frequency range of UV-A light, UV-B light, UV-C light or combinations thereof, at a temperature between about 20° C. and 40° C., at a moisture content between about 50% and about 90%, and for a period of time between 30 minutes and 12 hours.

16. A method comprising:
irradiating one or more raw fungus materials in a UV treating unit with UV light having a frequency or a frequency range of UV-A light, UV-B light, UV-C light or combinations thereof, at a temperature between about 20° C. and 40° C., at a moisture content between about 50% and about 90%, and for a period of time between 30 minutes and 12 hours to form a UV treated fungus material,
drying the UV treated fungus material at a temperature, at a pressure, and for a time sufficient to dry the UV treated fungus material to form a dried, UV treated fungus material, and
comminuting the dried, UV treated fungus material to form a powdered, dried, UV treated fungus material.

17. The method of claim 16, further comprising:
forming the powdered, dried, UV treated fungus material into a dose format or delivery system.

18. The method of claim 16, further comprising:
packaging the dose formats or delivery systems into user packages including a plurality of individual dose formats or delivery systems.

19. The method of claim 16, further comprising:
packaging the user packages into bulk packages for shipping.
